(19) United States
(12) Patent Application Publication
(10) Pub. No.: US 2024/0277600 A1
(43) Pub. Date: Aug. 22, 2024

(54) Water-In-Oil Emulsion Cosmetic

(71) Applicant: JO COSMETICS CO., LTD., Tokyo (JP)

(72) Inventor: Yasuhiro Nagata, Tokyo (JP)

(73) Assignee: JO COSMETICS CO., LTD., Tokyo (JP)

(21) Appl. No.: 18/566,196

(22) PCT Filed: Jun. 1, 2022
(86) PCT No.: PCT/JP2022/022245

§ 371 Date: Dec. 1, 2023

(30) Foreign Application Priority Data
Jun. 4, 2021 (JP) 2021-094500

(51) Int. Cl. A61K 8/891; A61K 8/06; A61K 8/37; A61K 8/58; A61K 8/92; A61Q 1/02; A61Q 17/04

(52) U.S. Cl. CPC A61K 8/891; A61K 8/064; A61K 8/375; A61K 8/585; A61K 8/92; A61Q 1/02; A61Q 17/04; A61K 2800/95

(57) ABSTRACT

A water-in-oil emulsion cosmetic containing: 1 to 20% by mass of a trimethylsiloxysilicate that has a viscosity display value of 500 to 130 [(mPa·s)×(g/cm³)] when being dissolved in decamethylcyclopentasiloxane at a concentration of 50% by mass (A); 10 to 60% by mass of a volatile oil (B); 0.2 to 10% by mass of a nonionic surfactant having an HLB of 2 to 8 (C); and 0 to 10% by mass of a nonvolatile liquid oil (D), wherein a ratio by mass of an amount of the component (A) to a total amount of the components (C) and (D) represented by [(A)/{(C)+(D)}] is 1 to 3. Trimethylsiloxysilicate of the component (A) may be a mixture of trimethylsiloxysilicate having a viscosity display value of more than 500 [(mPa·s)×(g/cm³)] (X) and trimethylsiloxysilicate having a viscosity display value of less than 130 {(mPa·s)×(g/cm³) (Y).

### TECHNICAL FIELD

The present invention relates to a water-in-oil emulsion cosmetic suitable as a make-up cosmetic or sunscreen cosmetic.

### BACKGROUND TECHNOLOGY

A water-in-oil emulsion cosmetic (W/O emulsion cosmetic) having an oil phase as a continuous phase and an aqueous phase as a dispersed phase generally is advantageous in water resistance and persistence of cosmetic film as compared with an oil-in-water emulsion cosmetic (O/W emulsion cosmetic) that has an aqueous phase as a continuous phase and an oil phase as a dispersed phase. Hence, many of makeup cosmetics and sunscreen cosmetics are marketed as a commodity of this dosage form.

In order to further improve water resistance and persistence of cosmetic film of the W/O emulsion cosmetic, incorporation of a film-forming agent that is capable of forming a film on the skin has been conventionally studied, and a technique using trimethylsiloxysilicate as the film-forming agent has been proposed.

A cosmetic containing trimethylsiloxysilicate has improved water resistance and persistence of cosmetic film. However, since a film of trimethylsiloxysilicate generally is high in hardness and brittle, the cosmetic is insufficient in feeling in use. In addition, the cosmetic has a drawback that a crack tends to occur due to movement of a cosmetic film. In order to solve such problems, a combination of trimethylsiloxysilicate and other components has been studied. For example, Patent Document 1 describes that a W/O emulsion cosmetic containing trimethylsiloxysilicate, a partially crosslinked polyether-modified organopolysiloxane polymer and an acrylic silicone-based graft copolymer is excellent in water resistance, oil resistance, adhesion to the skin, and persistence of cosmetic effects.

Patent Document 2 describes that a W/O cosmetic containing (A) trimethylsiloxysilicate, (B) polyalkylsilsesquioxane, (C) an acrylic-modified silicone and (D) a long chain alkyl-modified silicone is excellent in feeling use and stability.

As described above, various investigations have been made to overcome the drawbacks due to using trimethylsiloxysilicate as a film-forming agent by combining it with other components. However, as for flexibility of cosmetic film and color transfer resistance, that is, a property that a cosmetic does not cause secondary adhesion (color transfer) to clothing, masks, or the like, no cosmetic having sufficient performance has yet been obtained.

### PRIOR ART DOCUMENT

### Patent Document

Patent Document 1: Japanese Laid-open Patent Publication No. H10-236917

Patent Document 2: Japanese Laid-open Patent Publication No. 2018-095617

### DISCLOSURE OF THE INVENTION

### Problems to be Solved by the Invention

The present invention was completed based on such a background art, and an object thereof is to provide a W/O emulsion cosmetic that is excellent in flexibility of cosmetic film and color transfer resistance.

### Means Used to Solve the Problems

As a result of intensive studies to solve the problems mentioned above, the present inventors have found that a W/O emulsion cosmetic containing trimethylsiloxysilicate with a specific viscosity value exhibits excellent flexibility of cosmetic film and color transfer resistance. Thus, the present invention was completed.

According to the present invention, there is provided a water-in-oil emulsion cosmetic containing 1 to 20% by mass of trimethylsiloxysilicate that has a viscosity display value of 500 to 130 [(mPa·s)×(g/cm³)] when being dissolved in decamethylcyclopentasiloxane at a concentration of 50% by mass (A) 10 to 60% by mass of a volatile oil (B), 0.2 to 10% by mass of a nonionic surfactant having an HLB of 2 to 8 (C), and 0 to 10% by mass of a nonvolatile liquid oil (D), wherein a ratio by mass of an amount of the component (A) to a total amount of the components (C) and (D), that is, [(A)/{(C)+(D)}], is 1 to 3.

### Effect of the Invention

The water-in-oil emulsion cosmetic of the present invention is excellent in flexibility of cosmetic film and color transfer resistance.

### Embodiment for Carrying Out of the Invention

The W/O emulsion cosmetic of the present invention contains, as essential components, trimethylsiloxysilicate having a viscosity display value of 500 to 130 [((mPa·s)×(g/cm³)] when being dissolved in decamethylcyclopentasiloxane at a concentration of 50% by mass (A), a volatile oil (B) and a nonionic surfactant having an HLB of 2 to 8 (C). Each component will be described in detail below.

### (A: Trimethylsiloxysilicate)

Trimethylsiloxysilicate used as the component (A) of the present invention is a compound having a crosslinked structure that has a siloxane structure as a main skeleton, and is a silicone resin composed of SiO₂ units and (CH₃)₃ SiO_{0.5} units. This is formulated to improve water resistance and persistence of cosmetic film to water-in-oil emulsions.

Physical properties of trimethylsiloxysilicate depend on its molecular weight. For example, hardness and flexibility of a film formed by trimethylsiloxysilicate vary depending on its molecular weight. On the other hand, it is known that a viscosity of a polymer solution generally increases as a molecular weight of the polymer increases. Hence, in the present invention, as an index representing a molecular weight of trimethylsiloxysilicate, a viscosity display value measured at 25° C. by a vibrating viscometer of a decamethylcyclopentasiloxane solution having a concentration of 50% by mass of trimethylsiloxysilicate is adopted. Hereinafter, the viscosity display value that is obtained under the specified condition mentioned above is sometimes simply referred to as “viscosity display value”. It is noted that the viscosity display value is a value measured by the following method.

### (Method for Measuring a Viscosity Display Value of Trimethylsiloxysilicate)

Sample: Trimethylsiloxysilicate is dissolved in decamethylcyclopentasiloxane in a concentration of 50% by mass to make a measurement sample. If a commercially available raw material is a solution of decamethylcyclopentasiloxane, which has an INCI name of cyclopentasiloxane, with a trimethylsiloxysilicate concentration of 50% by mass, it is used as it is, and if the commercially available raw material is a solution of any other volatile solvent, after completely volatilizing the solvent, a sample is prepared by diluting with decamethylcyclopentasiloxane. When a plurality of raw materials for trimethylsiloxysilicate are used in admixture, a decamethylcyclopentasiloxane solution having a trimethylsiloxysilicate concentration of 50% by mass is prepared in consideration of each solvent and a mixing ratio. Measurement instrument: Tuning fork vibration viscometer SV-10 (A & D Company Ltd.)
Measurement temperature: 25° C.
Measurement condition: 30 Hz
Measurement time: 30 seconds
Viscosity display value: Since physical quantities measured by the measurement instrument mentioned above are detected as a product of viscosity×density on the principle of measurement, a unit of the viscosity display value is expressed as [(mPa·s)×(g/cm³)].

When each viscosity display value of commercially available trimethylsiloxysiliciates is measured according to the method described above, each value expressed as [(mPa·s)×(g/cm³)] is as follows:
Trade name KF-9021 (Shin-Etsu Chemical Co. Ltd.): 6,290
Trade name DOWSIL MQ-1600 (Dow Toray Inc.): 850
Trade name BELSIL TMS 803 (Wacker Asahikasei Silicone Co., Ltd.): 529
Trade name X-21-5250 (Shin-Etsu Chemical Co. Ltd.): 37
Trade name X-21-5249 (Shin-Etsu Chemical Co. Ltd.): 71
Trade name KF-7312 J (Shin-Etsu Chemical Co. Ltd.): 128

The viscosity display value of the component (A) is an important requirement of the present invention and it is necessary to use trimethylsiloxysiliciate having a viscosity display value of 500 to 130 [(mPa·s)×(g/cm³)]. It is preferred to use trimethylsiloxysiliciate having a viscosity display value of 450 to 150 [(mPa·s)×(g/cm³)]. Any trimethylsiloxysilicate having a viscosity display value within this range can be used alone or in combination of two or more, and it is possible to use in combination of a high-viscosity trimethylsiloxysilicate and a low-viscosity trimethylsiloxysilicate so as to make a mixture having a viscosity display value within this range. When the viscosity display value is greater than 500 [(mPa·s)×(g/cm³)], a cosmetic film loses flexibility and tends to tear due to poor flexibility, resulting in poor cosmetic persistence. Also, the cosmetic film tends to give a feeling in tightness. Conversely, when the viscosity display value is smaller than 130 [(mPa·s)×(g/cm³)], color transfer resistance decreases due to lowering fastness of cosmetic film, and the cosmetic film tends to give stickiness.

A content of the component (A) is 1 to 20% by mass, preferably 2 to 15% by mass, more preferably 5 to 12% by mass, based on the whole cosmetic. When the content of the component (A) is too small, secondary adhesion is likely to occur. When too large, it is difficult to spread the cosmetic to the skin, and the cosmetic gives feeling in tightness upon using it. It is noted that “% by mass” of each component in the following description is also based on the whole cosmetic.

In the present invention, the component (A) may be a mixture prepared by combining trimethylsiloxysilicate with a viscosity display value of more than 500 [(mPa·s)×(g/cm³)] and trimethylsiloxysilicate with a viscosity display value of less than 130 [(mPa·s)×(g/cm³)] so as to have a predetermined viscosity display value. Hereinafter, the former is referred to as component (X), and the latter is referred to as component (Y). In the case of such a combination, it is possible to easily achieve both color transfer resistance and flexibility of cosmetic film. The component (X) preferably has a viscosity display value of 500 to 7,000 [(mPa·s)×(g/cm³)] and the component (Y) preferably has a viscosity display value of 30 to 100 [(mPa·s)×(g/cm³)]. Incidentally, even if a pasty film-forming agent such as polypropylsilsesquioxane is used in combination with the component (X) for the purpose of imparting flexibility to a film formed by the component (X), the effects of the present invention cannot be achieved.

A blending ratio of the component (X) and the component (Y) is not particularly limited as long as a viscosity display value of a mixture composed of the component (X) and the component (Y) is 500 to 130 [(mPa·s)×(g/cm³)]. A blending ratio by mass of the component (X) to the component (Y), that is, (X)/(Y) is preferably 0.5 to 4, more preferably 0.7 to 3, particularly preferably 1 to 2. When a blending ratio of the (X) component increases, a cosmetic film tends to tear, and conversely, when a blending ratio of the component (Y) increases, color transfer resistance of cosmetic film tends to decrease.

Examples of trimethylsiloxysilicate available commercially include X-21-5249, X-21-5249 L, X-21-5250, X-21-5250 L, X-21-5616, X-21-5595, KF-7312 J, KF-7312 F, KF-9021, and KF-9021 L, any of which is available from Shin-Etsu Chemical Co., Ltd., RSN-0749, 593 Fluid, and MQ-1600, any of which belongs to DOWSIL® series and is available from Dow Toray Inc., SR-1000, SS 4230, SS 4267, and SILSOFT 74, any of which is available from Momentive Performance Materials, and BELSIL® TMS 803 available from Wacker Asahikasei Silicone Co. Ltd.

A volatile oil of the component (B) used in the present invention is not limited as long as it is used in conventional cosmetics. Usually, the volatile oil has a boiling point at normal pressure of 60 to 260° C., preferably 100 to 220° C. Examples thereof include cyclic silicone oils such as octamethylcyclotetrasiloxane, decamethylcyclopentasiloxane, and dodecamethylcyclohexasiloxane, silicone oils such as low molecular weight dimethicone and methyltrimethicone, hydrocarbon oils such as isododecane, isohexadecane, and a hydrocarbon oil such as a saturated isoparaffinic hydrocarbon oil containing a compound with carbon atoms of 8 to 16 as a main component (for example, hydrogenated polybutene). Examples of commercially available products include KF-994(octamethylcyclotetrasiloxane), KF-995 (decamethylcyclopentasiloxane), KF-96A-1cs (octamethyltrisiloxane), KF-96L-1.5cs (decamethyltetrasiloxane), TMF-1.5(methyltrimethicone), any of which is available from Shin-Etsu Chemical Co. Ltd., Marukasol R (isododecane) which is available from Maruzen Petrochemical Co., Ltd., IP solvent 1620 (hydrogenated polyisobutene) available from Idemitsu Kosan Co., Ltd., and IP solvent 2028 (hydrogenated polyisobutene) available from Idemitsu Kosan Co., Ltd. Of these, octamethyltrisiloxane, methyltrimethicone, isododecane, and hydrogenated polyisobutene containing a compound with carbon atoms of 8 to 16 as a main component are preferably used in view of storage stability and volatilization rate.

A content of the component (B) is 10 to 60% by mass, preferably 20 to 55% by mass, more preferably 35 to 50% by mass relative to the whole cosmetic. If the content is too small, it becomes difficult to spread a cosmetic when applied to the skin, resulting in being difficult to apply the cosmetic uniformly. On the other hand, when this content is excessively large, persistence of cosmetic film deteriorates because a cosmetic film becomes thin.

### (C: Nonionic Surfactant Having an HLB of 2 to 8).

The component (C) used in the present invention is a nonionic surfactant having an HLB of 2 to 8. Examples thereof include glycerin fatty acid esters, polyglycerin fatty acid esters, polyoxyethylene glycerin fatty acid esters, propylene glycol fatty acid esters, polyoxyethylene polyoxypropylene glycol, sorbitan fatty acid esters, polyoxyethylene sorbitan fatty acid esters, polyoxyethylene castor oil, polyoxyethylene hydrogenated castor oil, polyoxyethylene hydrogenated castor oil fatty acid esters, polyethylene glycol fatty acid esters, alkyl glyceryl ethers, alkyl polyglyceryl ethers, polyoxyethylene alkyl ethers, polyoxyethylene alkyl ether fatty acid esters, alkyl alkanolamides, polyoxyethylene alkyl amines, fatty acid alkanolamides, polyether-modified silicones, glycerin-modified silicones and the like. Of these, polyether-modified silicones and polyglycerin fatty acid esters are preferred.

The component (C) is a surfactant having an HLB of 2 to 8, preferably 2 to 5 in view of stably making a W/O emulsion cosmetic. Here, HLB (Hydrophilic-Lipophilic Balance) indicates a molecular weight of a hydrophilic group portion in the total molecular weight of a surfactant, and is determined according to Griffin's formula.

A content of the component (C) is necessary to be 0.2 to 10% by mass in the W/O emulsion cosmetic in view of making a stable formulation by emulsifying an aqueous phase in an oil phase, and it is preferably 0.5 to 10% by mass, more preferably 2 to 8% by mass. The surfactant of the component (C) may be used alone or in combination of two or more.

In addition to the components (A), (B) and (C) mentioned above, a non-volatile liquid oil (D) may be added in a content of 10% by mass or less. Preferably, the content of the component (D) is 0.2 to 7% by mass. Incorporation of the component (D) improves flexibility of cosmetic film.

### (D: Non-Volatile Liquid Oil)

In the present invention, “non-volatile liquid oil” means an oil that is liquid or pasty at normal temperature and normal pressure, and does not include a volatile oil with a boiling point of 260° C. or less and a water-soluble component. A lipophilic nonionic surfactant not corresponding to the component (B) is also included in the “non-volatile liquid oil” as long as it meets the conditions mentioned above.

The non-volatile liquid oil used as the component (D) in the present invention is not particularly limited as long as it is used in conventional cosmetics, and may be any of animal oil, vegetable oil and synthetic oil. Examples of the non-volatile liquid oil include esters such as triethylhexoin, caprylic/capric triglyceride, neopentyl glycol dicaprate, isononyl isononanoate, isotridecyl isononanoate, ethylhexyl palmitate, isopropyl myristate, isopropyl palmitate, octyldodecyl myristate, diisostearyl malate, oligomer ester of dimer acid and dimer diol, pentaerythritol tetraisostearate, diglyceryl tetraisostearate, cetyl isooctanoate, cholesterol fatty acid ester, and jojoba oil; hydrocarbons such as polybutene, polyisobutylene, heavy liquid isoparaffin, liquid paraffin, alpha-olefin oligomer, squalane, and petrolatum; oils and fats such as olive oil, castor oil, mink oil, macadamia nut oil; fatty acids such as isostearic acid and oleic acid; higher alcohols such as oleyl alcohol and isostearyl alcohol; silicone oils such low polymerization degree dimethylpolysiloxane, high polymerization degree dimethylpolysiloxane, methylphenylpolysiloxane, caprylyltrimethicone, crosslinked organopolysiloxane, fluorine-modified polysiloxanes; fluorine-based oils such as perfluoropolyether; lanolin derivatives such as lanolin, lanolin acetate, lanolin fatty acid isopropyl, lanolin alcohol; and the like.

Each content of the component (C) and the component (D) is 0.2 to 10% by mass and 0 to 10% by mass respectively, and a total content of both components is preferably 15% by mass or less, more preferably 3 to 10% by mass. When the total content of both components is excessively large, flexibility of a film formed by the component (A) increases, and stickiness and color transfer tend to occur. A ratio of an amount of the component (A) to a total amount of the component (C) and the component (D), that is [(A)/{(C)+(D)}], is preferably 1 to 3 or less. If it is smaller than 1, color transfer resistance tends to be reduced and stickiness tends to be felt. If it is greater than 3, flexibility of cosmetic film tends to be reduced. In both cases, cosmetic persistence is adversely affected.

The W/O emulsion cosmetic of the present invention contains water as component (E). The water may be one that is known to be suitable for manufacturing cosmetics, and examples thereof include ion-exchanged water and purified water such as distilled water. A content of the water in the cosmetic is not particularly limited, but is preferably 5 to 80% by mass, more preferably 10 to 50% by mass, furthermore preferably 15 to 40% by mass based on the total mass of the cosmetic.

The water-in-oil emulsion cosmetic of the present invention may contain other ingredients formulated in conventional cosmetics. Examples of such an ingredient include oil-soluble resins other than the component (A), solid oils, powders, water-soluble polymers, alcohols, clay minerals, resins, humectants, preservatives, antimicrobial agents, perfumes, salts, antioxidants, pH adjusters, chelating agents, refreshing agents, anti-inflammatory agents, skin-beautifying ingredients, vitamins, amino acids, nucleic acids, clathrate compounds, and the like.

Examples of the oil-soluble resin other than the component (A) include silicone-based resins such as partially crosslinked organopolysiloxane, trimethylsiloxysilylcarbamoyl pullulan, polymethylsilsesquioxane, polypropylsilsesquioxane, fluorine-modified silicone, acrylic modified silicone, silicone dendrimer-modified resin compounds; rosin acid-based resins such as pentaerythrityl hydrogenated rosinate and hydrogenated glyceryl dehydroabietate; candelilla resin, polyvinyl acetate-based resin, polyvinyl isobutyl ether, polyisobutylene, and the like. The candelilla resin is a resin component obtained via fractional extraction of candelilla wax with an organic solvent, wherein the resin content is preferably at least 65% by mass, more preferably at least 85% by mass. When the oil-soluble resin other than the component (A) is not solid at normal temperature and is pasty, it is classified herein as the non-volatile liquid oil (D). Examples of the solid oils include animal waxes, vegetable waxes, mineral waxes, silicone waxes, and synthetic waxes, any of which have a melting point of 50° C. or higher.

The powders are formulated for the purpose of adjusting feeling in touch and imparting a makeup effect or the like. As long as they are used in conventional cosmetics, they are not particularly limited by a shape such as a plate-like, spindle-like, and needle-like, and a particle structure such as particle size, porous, and non-porous, and the like. They may be any of inorganic powders, glittering powders, organic powders, pigments, and composite powders.

Specific examples of the powders used include inorganic powders such as titanium oxide, zinc oxide, zirconium oxide, cerium oxide, iron oxide, Ferric Ferrocyanide, ultramarine, silicic anhydride, magnesium carbonate, calcium carbonate, aluminum hydroxide, chromium hydroxide, carbon black, aluminum silicate, magnesium silicate, aluminum magnesium silicate, mica, smectite, bentonite, kaolin, synthetic mica, synthetic sericite, sericite, talc, silicon carbide, barium sulfate, and boron nitride; glittering powders such as bismuth oxychloride, titanated mica, iron oxide-coated mica, iron oxide-coated titanated mica, organic pigment-coated titanated mica, and aluminum powder; organic powders such as magnesium stearate, zinc stearate, N-acyl_lysine, polystyrene, nylon, polymethyl methacrylate, polymethylsilsesquioxane powder, and organopolysiloxane elastomer powder.

If needed, the powders may be surface-treated with a known surface-treating agent, such as inorganic compounds (for example, alumina, silica, and iron oxide), fluorine compounds, silicone compounds, phospholipids, phospholipid derivatives, metallic soaps, waxes, surfactants, fats, and hydrocarbons. These may be used alone or in combination of two or more.

The water-in-oil emulsion cosmetic of the present invention can be prepared by conventional methods. A dosage form may be any of creamy, milky, or solid. This cosmetic is used not only as a makeup cosmetic such as foundation, base, concealer, blush, eye shadow, and lip color, and as a sunscreen cosmetic, but also as other cosmetics that require persistence of cosmetic film. Specific examples of other cosmetics include skin care cosmetics such as hand cream, BB cream, and CC cream, and hair cosmetics.

### EXAMPLE

Hereinafter, the present invention will be described more specifically with reference to Examples and Comparative Examples, but the present invention is not limited by these Examples. A compounding amount in formulations in the following description is expressed as “% by mass” relative to the total amount unless otherwise noted.

Evaluation items and evaluation methods of W/O emulsion cosmetics in the following Examples and Comparative Examples are as follows.

### (Evaluation Item)

a: Spreadability
b: Non-stickiness-I
c: Degree of feeling in tightness
d: Cosmetic persistence
e: Non-stickiness-II
f: Color transfer resistance
g: Flexibility of cosmetic film

### (Evaluation Method for the Evaluation Items a to d)

For each of the evaluation items a to d, 10 evaluators applied a sample to their face and evaluated sensorily in four stages according to the following evaluation criteria(score):

### (Score)

5: Very good.
4: Good
3: Ordinary
2: Poor
1: Very poor

### (Evaluation Criteria)

A: Average value of scores by 10 evaluators is 4.0 or more.
B: Average value of scores by 10 evaluators is 3.5 or more and less than 4.0.
C: Average value of scores by 10 evaluators is 2.5 or more and less than 3.5.
D: Average value of scores by 10 evaluators is less than 2.5.

### (Evaluation Method for the Evaluation Items e to g)

After preparing the water-in-oil emulsion cosmetics of Examples and Comparative Examples, 0.05 g of a sample was uniformly applied on a surface with a size of 30 mm×50 mm of a rectangular test piece with a size of 30 mm×70 mm made by cutting a urethane artificial skin for evaluation (SKIN MODEL No. 77 2T #BLACK manufactured by Beaulux Co., Ltd.), and dried at room temperature for 45 minutes or more to make a test piece. Each evaluation item was evaluated using the test piece.

### (Evaluation Method: Non-Stickiness-II)

A tissue paper (TANOSEE tissue paper available from OTSUKA CORPORATION) was placed on a test piece, and a degree of easiness for peeling off the tissue paper when lightly pressed and when strongly pressed, respectively was evaluated according to the following evaluation criteria.

### (Evaluation Criteria)

A: Can be peeled off without resistance even when pressed strongly.
B: Can be peeled off without resistance when pressed lightly, but there is a feeling of resistance for peeling off the tissue paper when pressed strongly.
C: When pressed lightly, there is a feeling of resistance for peeling off the tissue paper, while when pressed strongly, the tissue paper is torn and a part thereof remains on the surface of the test piece.

### (Evaluation Method: Color Transfer Resistance)

A tissue paper was placed on a test piece, and a degree of color transfer when strongly pressed was evaluated according to the following evaluation criteria.

### (Evaluation Criteria)

A: No color transfer is observed.
B: A slight color transfer is observed.
C: An apparent color transfer is observed.

### (Evaluation Method: Flexibility of Cosmetic Film)

After a test piece was elongated by 150% in the long side direction, a power for elongation was released for returning the elongated test piece to the original form. A state of a cosmetic film after the power for elongation was released was evaluated according to the following evaluation criteria.

### (Evaluation Criteria)

A: No crack occurs in the cosmetic film.
B: Cracks occur in the cosmetic film.
C: Cracks occur in the cosmetic film and the cosmetic film is peeled off

### Examples 1 to 2 and Comparative Examples 1 to 3

### (Liquid Foundation)

A liquid foundation having the formulation shown in Table 1 was prepared according to the following manufacturing procedure and evaluated for a performance as a cosmetic according to the evaluation method described above. In addition, each viscosity display value on the component (X), the component (Y) and a mixture of both components was measured according to the methods described above. Formulations, viscosity display values, and evaluation results are shown in Table 1. Since a commercially available product that is an equivalent mixture with decamethylcyclopentasiloxane was used as trimethylsiloxysilicate of the components (X) and (Y) shown in Table 1, a content of trimethylsiloxysilicate in the formulations shown in Table 1 indicates a mass of the mixture.

### (Manufacturing Procedure)

(1) The ingredients 1 to 12 were mixed to prepare an oil phase (a)
(2) The ingredients 13 to 16 were mixed to prepare an aqueous phase (b).
(3) The aqueous phase (b) was mixed with the oil phase (a) little by little at room temperature to prepare a cosmetic.

TABLE 1

| | | | Example | | Comparative Example | | |
|---|---|---|---|---|---|---|---|
| Ingredient | | | 1 | 2 | 1 | 2 | 3 |
| 1 | B | Decamethylcyclopentasiloxane | Balance | Balance | Balance | Balance | Balance |
| 2 | | Dimethicone-treated titanium dioxide | 12.50 | 12.50 | 12.50 | 12.50 | 12.50 |
| 3 | | Dimethicone-treated iron oxides | 1.20 | 1.20 | 1.20 | 1.20 | 1.20 |
| 4 | C | PEG-10dimethicone(※1) | 2.60 | 2.60 | 2.60 | 2.60 | 2.60 |
| 5 | | Disteardimonium hectorite(※2) | 0.40 | 0.40 | 0.40 | 0.40 | 0.40 |
| 6 | X | Trimethylsiloxysilicate KF-9021(※3) | 11.00 | 9.00 | | 12.00 | 6.00 |
| 7 | Y | Trimethylsiloxysilicate X-21-5250(※4) | 3.00 | 9.00 | | | 12.00 |
| 8 | Y | Trimethylsiloxysilicate X-21-5249(※5) | 2.00 | | | | |
| | Y | Trimethylsiloxysilicate KF-7312J(※6) | 2.00 | | 18.00 | 6.00 | |
| 9 | D | Triethylhexanoin | 0.50 | 0.50 | 0.50 | 0.50 | 0.50 |
| 10 | C | Polyglyceryl-2 diisostearate(※7) | 1.50 | 1.50 | 1.50 | 1.50 | 1.50 |
| 11 | | Polymethylsilsesquioxane powder(※8) | 1.00 | 1.00 | 1.00 | 1.00 | 1.00 |
| 12 | | Dimethicone-treated mica | 2.00 | 2.00 | 2.00 | 2.00 | 2.00 |
| 13 | | purified water | 23.00 | 23.00 | 23.00 | 23.00 | 23.00 |
| 14 | | Magnesium sulfate | 1.00 | 1.00 | 1.00 | 1.00 | 1.00 |
| 15 | | Butylene glycol | 5.00 | 5.00 | 5.00 | 5.00 | 5.00 |
| 16 | | Phenoxyethanol | 0.50 | 0.50 | 0.50 | 0.50 | 0.50 |
| | | Viscosity display value of component (X), | 426 | 160 | 128 | 1320 | 78 |
| | | component (Y) or a mixture of both components | | | | | |
| | | Content of component (A) | 9.00 | 9.00 | | | |
| | | Total content of component (C) and component (D) | 4.60 | 4.60 | 4.60 | 4.60 | 4.60 |
| | | (A)/[(C) + (D)] | 1.96 | 1.96 | | | |
| Evalu- | | Spreadability | A | A | A | B | A |
| ation | | Non-stickiness-I | A | A | A | A | B |
| | | Degree of feeling in tightness | A | A | A | B | A |
| | | Cosmetic persistence | A | A | B | B | B |
| | | Non-stickiness-II | A | A | A | A | B |
| | | Color transfer resistance | A | A | C | B | C |
| | | Flexibility of cosmetic film | A | A | A | C | A |

(※1)Trade name KF-6017 (Shin-Etsu Chemical Co., Ltd.)
(※2)Trade name BENTONE 38V (Elementis PLC)
(※3)Trade name KF-9021 (Shin-Etsu Chemical Co., Ltd.) Mixture having a ratio by mass of trimethylsiloxysilicate to decamethylcyclopentasiloxane of 50/50
(※4)Trade name X-21-5250 (Shin-Etsu Chemical Co., Ltd.) Mixture having a ratio by mass of trimethylsiloxysilicate to decamethylcyclopentasiloxane of 50/50
(※5)Trade name X-21-5249 (Shin-Etsu Chemical Co., Ltd.) Mixture having a ratio by mass of trimethylsiloxysilicate to decamethylcyclopentasiloxane of 50/50
(※6)Trade name KF-7312J(Shin-Etsu Chemical Co., Ltd.) Mixture having a ratio by mass of trimethylsiloxysilicate to decamethylcyclopentasiloxane of 50/50
(※7)Trade name COSMOL 42V(The Nissin Oillio Group Co., Ltd.)
(※8)Trade name TOSPEARL 2000B * (Momentive Performance Materials)

As apparent from the results shown in Table 1, the water-in-oil emulsion cosmetic of the present invention was excellent in spreadability, non-stickiness, less feeling in tightness, cosmetic persistence, and flexibility of cosmetic film (refer to Examples 1 to 2). On the other hand, the cosmetic of Comparative Example 2 containing the component (A) having a viscosity display value of more than 500 [(mPa·s)×(g/cm³)] was inferior in flexibility of cosmetic film, and the cosmetics of Comparative Example 1 and Comparative Example 3 containing the component (A) having a viscosity display value of less than 130 [(mPa·s)×(g/cm³)] were inferior in color transfer resistance and were insufficient in cosmetic persistence. In particular, the cosmetic of Comparative Example 3 containing trimethylsiloxysilicate having a viscosity display value of less than 100 [(mPa·s)×(g/cm³)] was also inferior in non-stickiness.

### Examples 3 to 5 and Comparative Examples 4 to 5

### (Liquid Foundation)

A liquid foundation having the formulation shown in Table 2 was prepared according to the following manufacturing procedure and evaluated according to the evaluation method described above. The results are also shown in Table 2. Trimethylsiloxysilcate used in Comparative Example 4 shown in Table 2 is composed of trimethylsiloxysilcate only, and contains no decamethylcyclopentasiloxane.

### (Manufacturing Procedure)

(1) The ingredients 1 to 12 were mixed to prepare an oil phase (a)
(2) The ingredients 13 to 16 were mixed to prepare an aqueous phase (b).
(3) The aqueous phase (b) was mixed with the oil phase (a) little by little at room temperature to prepare a cosmetic.

TABLE 2

| | | | Example | | | Comparative Example | |
|---|---|---|---|---|---|---|---|
| Ingredient | | | 3 | 4 | 5 | 4 | 5 |
| 1 | B | Decamethylcyclopentasiloxane | Balance | Balance | Balance | Balance | Balance |
| 2 | | Dimethicone-treated titanium dioxide | 12.50 | 12.50 | 12.50 | 12.50 | 12.50 |
| 3 | | Dimethicone-treated iron oxides | 1.20 | 1.20 | 1.20 | 1.20 | 1.20 |
| 4 | C | PEG-10dimethicone(※1) | 2.60 | 2.60 | 2.60 | 2.60 | 2.60 |
| 5 | | Disteardimonium hectorite(※2) | 0.40 | 0.40 | 0.40 | 0.40 | 0.40 |
| 6 | X | Trimethylsiloxysilicate KF-9021(※3) | 7.00 | 13.00 | 12.00 | | 15.00 |
| | X | Trimethylsiloxysilicate MQ-1600(※9) | | | | 6.00 | |
| 7 | Y | Trimethylsiloxysilicate X-21-5250(※4) | 5.00 | 7.00 | 6.00 | 4.00 | 15.00 |
| 8 | C, D | 670 Fluid(※10) | | | 6.00 | 8.00 | |
| 9 | D | Triethylhexanoin | 0.50 | 0.50 | 0.50 | 0.50 | 0.50 |
| 10 | C | Polyglyceryl-2 diisostearate(※7) | 1.50 | 1.50 | 1.50 | 1.50 | 1.50 |
| 11 | | Polymethylsilsesquioxane powder(※8) | 1.00 | 1.00 | 1.00 | 1.00 | 1.00 |
| 12 | | Dimethicone-treated mica | 2.00 | 2.00 | 2.00 | 2.00 | 2.00 |
| 13 | | purified water | 23.00 | 23.00 | 23.00 | 23.00 | 23.00 |
| 14 | | Magnesium sulfate | 1.00 | 1.00 | 1.00 | 1.00 | 1.00 |
| 15 | | Butylene glycol | 5.00 | 5.00 | 5.00 | 5.00 | 5.00 |
| 16 | | Phenoxyethanol | 0.50 | 0.50 | 0.50 | 0.50 | 0.50 |
| | | Viscosity display value of mixture of | 230 | 330 | 426 | 237 | 160 |
| | | components (X) and (Y), i.e. component (A) | | | | | |
| | | Content of component (A) | 6.00 | 10.00 | 9.00 | 8.00 | 15.00 |
| | | Total content of components (C) and (D) | 4.60 | 4.60 | 7.60 | 8.60 | 4.60 |
| | | (A)/[(C) + (D)] | 1.30 | 2.17 | 1.18 | 0.93 | 3.26 |
| Evalu- | | Spreadability | A | A | A | A | B |
| ation | | Non-stickiness-I | A | A | A | B | A |
| | | Degree of feeling in tightness | A | A | A | A | B |
| | | Cosmetic persistence | A | A | A | B | B |
| | | Non-stickiness-II | A | A | A | C | A |
| | | Color transfer resistance | A | A | A | B | A |
| | | Flexibility of cosmetic film | A | A | A | A | C |

(※9)Trade name DOWSIL ® MQ-1600 (Dow Toray Co., Ltd.): 100% Product made of trimethylsiloxysilicate only
(※10)Trade name DOWSIL ® 670 Fluid (Dow Toray Co., Ltd.) Mixture having a ratio by mass of polypropylsilsesquioxane to decamethylcyclopentasiloxane of 50/50

As apparent from the results shown in Table 2, the water-in-oil emulsion cosmetic of the present invention was excellent in spreadability, non-stickiness, less feeling in tightness, cosmetic persistence, and flexibility of cosmetic film (refer to Examples 3 to 5). On the other hand, the cosmetic of Comparative Example 4, in which a ratio by mass of the component (A) to the sum of the component (C) and the component (D), that is, [(A)/[(C)+(D)] is less than 1, was inferior in non-stickiness, and the cosmetic of Comparative Example 5, in which the ratio by mass is more than 3, was inferior in flexibility of cosmetic film. Incidentally, when a pasty polypropylsilsesquioxane was used to impart flexibility to a film formed by the component (X), sufficient performance in terms of non-stickiness could not be obtained.

### Example 6

### (Liquid Foundation)

A liquid foundation having the formulation shown in Table 3 was prepared according to the following manufacturing procedure and evaluated according to the evaluation method described above. The results are also shown in Table 3.

### (Manufacturing Procedure)

(1) The ingredients 1 to 13 were mixed to prepare an oil phase (a)
(2) The ingredients 14 to 17 were mixed to prepare an aqueous phase (b).
(3) The aqueous phase (b) was mixed with the oil phase (a) little by little at room temperature to prepare a cosmetic.

TABLE 3

| Ingredient | | | Example6 |
|---|---|---|---|
| 1 | B | Isodecane | Balance |
| 2 | B | Dimethylpolysiloxane(2 mm²/s)(※11) | 8.00 |
| 3 | | Dimethicone-treated titanium dioxide | 12.50 |
| 4 | | Dimethicone-treated iron oxides | 1.20 |
| 5 | C | PEG-10dimethicone | 2.60 |
| 6 | | Disteardimonium hectorite | 0.40 |
| 7 | X | Trimethylsiloxysilicate MQ-1600(※9) | 6.00 |
| 8 | Y | Trimethylsiloxysilicate X-21-5249(※5) | 3.00 |
| 9 | Y | Trimethylsiloxysilicate KF-7312J(※6) | 3.00 |
| 10 | D | Triethylhexanoin | 0.50 |
| 11 | C | Polyglyceryl-2 diisostearate(※7) | 1.50 |
| 12 | | Polymethylsilsesquioxane powder(※8) | 1.00 |
| 13 | | Dimethicone-treated mica | 2.00 |
| 14 | | purified water | 23.00 |
| 15 | | Magnesium sulfate | 1.00 |
| 16 | | Butylene glycol | 5.00 |
| 17 | | Phenoxyethanol | 0.50 |
| | | Viscosity display value of mixture of | 270 |
| | | components (X) and (Y), i.e. component (A) | |
| | | Content of component (A) | 9.00 |
| | | Total content of components (C) and (D) | 4.60 |
| | | (A)/[(C) + (D)] | 1.96 |
| Evaluation | | Spreadability | A |
| | | Non-stickiness-I | A |
| | | Degree of feeling in tightness | A |
| | | Cosmetic persistence | A |
| | | Non-stickiness-II | A |
| | | Color transfer resistance | A |
| | | Flexibility of cosmetic film | A |

(※11)Trade name SILICONE KF-96L 2CS(Shin-Etsu Chemical Co., Ltd.)

As apparent from the results shown in Table 3, the liquid foundation of Example 6 was excellent in spreadability, non-stickiness, non-tightness, less feeling in tightness, cosmetic persistence, and flexibility of cosmetic film.

### Example 7

### (W/O Emulsion Sunscreen Cosmetic)

A liquid foundation having the formulation shown in Table 4 was prepared according to the following manufacturing procedure and evaluated according to the evaluation method described above. The results are also shown in Table 4.

### (Manufacturing Procedure)

(1) The ingredients 1 to 10 were mixed to prepare an oil phase (a)
(2) The ingredients 11 to 13 were mixed to prepare an aqueous phase (b).
(3) The aqueous phase (b) was mixed with the oil phase (a) little by little at room temperature to prepare a cosmetic.

TABLE 4

| | | | Example |
|---|---|---|---|
| Ingredient | | | 7 |
| 1 | B | Cyclopentasiloxane | Balance |
| 2 | | Hydrogen Dimethicone-treated fine titanium | 7.00 |
| | | didioxide | |
| 3 | | Hydrogen Dimethicone-treated fine zinc oxide | 13.00 |
| 4 | C | Sorbitan sesquiisostearate(※12) | 1.00 |
| 5 | C | PEG-9 Polydimethylsiloxyethyl | 1.00 |
| | | Dimethicone(※13) | |
| 6 | C | Lauryl PEG-9 Polydimethylsiloxyethyl | 1.00 |
| | | Dimethicone(※14) | |
| 7 | X | Trimethylsiloxysilicate KF-9021(※3) | 9.00 |
| 8 | Y | Trimethylsiloxysilicate X-21-5250(※4) | 9.00 |
| 9 | | Methyl methacrylate crosspolymer(※15) | 6.00 |
| 10 | D | Triethylhexanoin | 5.00 |
| 11 | | purified water | 30.00 |
| 12 | | Butylene glycol | 5.00 |
| 13 | | Phenoxyethanol | 0.50 |
| | | Viscosity display value of mixture of | 160 |
| | | components (X) and (Y), i.e. component (A) | |
| | | Content of component (A) | 9.00 |
| | | Total content of components (C) and (D) | 8.00 |
| | | (A)/[(C) + (D)] | 1.13 |
| Evalua- | | Spreadability | A |
| tion | | Non-stickiness-I | A |
| | | Degree of feeling in tightness | A |
| | | Cosmetic persistence | A |
| | | Non-stickiness-II | A |
| | | Color transfer resistance | A |
| | | Flexibility of cosmetic film | A |

(※12)Trade name NIKKOL SI-15RV(Nikko Chemical Co., Ltd.)
(※13)Trade name KF-6028(Shin-Etsu Chemical Co., Ltd.)
(※14)Trade name KF-6038(Shin-Etsu Chemical Co., Ltd.)
(※15)Trade name GANZPEARL GMX-6010(Aica Kogyo Company, Limited)

As apparent from the results shown in Table 4, the liquid foundation of Example 6 was excellent in spreadability, non-stickiness, less feeling in tightness, cosmetic persistence, and flexibility of cosmetic film.

### Example 8

### (W/O Emulsion Solid Base Cosmetic)

A water-in-oil emulsion solid base cosmetic shown in Table 5 was prepared according to the following manufacturing procedure and evaluated according to the evaluation method described above on items except for spreadability. The results are also shown in Table 5.

### (Manufacturing Procedure)

(1) The ingredients 1 to 13 were mixed at 80° C. to prepare an oil phase (a).
(2) The ingredients 14 to 16 were mixed to prepare an aqueous phase (b).
(3) After heating the aqueous phase (b) to 80° C., the aqueous phase (b) was mixed with the oil phase (a) little by little, and then the mixture obtained was cooled to room temperature to prepare a cosmetic.

TABLE 5

| | | | Example |
|---|---|---|---|
| Ingredient | | | 8 |
| 1 | B | Cyclopentasiloxane | Balance |
| 2 | | (Dimethicone/Vinyl Dimethicone) | 20.00 |
| | | crosspolymer/Cyclopentasiloxane = 5/95(※16) | |
| 3 | | Dimethicone-treated titanium dioxide | 1.00 |
| 4 | | Dimethicone-treated iron oxides | 0.10 |
| 5 | C | Lauryl PEG-9 Polydimethylsiloxyethyl | 2.50 |
| | | Dimethicone | |
| 6 | | Disteardimonium hectorite | 0.50 |
| 7 | X | Trimethylsiloxysilicate KF-9021(※3) | 9.00 |
| 8 | Y | Trimethylsiloxysilicate X-21-5250(※4) | 6.00 |
| 9 | D | Dimethylpolysiloxane(6 mm²s)(※17) | 3.00 |
| 10 | | Polymethylsilsesquioxane(※10) | 3.00 |
| 11 | | Lauroyl lysine(※18) | 1.50 |
| 12 | | Synthetic phlogopite | 2.00 |
| 13 | | Ceresin | 6.00 |
| 14 | | Purified water | 32.00 |
| 15 | | Sodium chloride | 1.00 |
| 16 | | Phenoxyethanol | 0.50 |
| | | Viscosity display value of mixture of | 270 |
| | | components (X) and (Y), i.e. component (A) | |
| | | Content of component (A) | 7.50 |
| | | Total content of components (C) and (D) | 5.50 |
| | | (A)/[(C) + (D)] | 1.36 |
| Evalua- | | Spreadability | A |
| tion | | Non-stickiness-I | A |
| | | Degree of feeling in tightness | A |
| | | Cosmetic persistence | A |
| | | Non-stickiness-II | A |
| | | Color transfer resistance | A |
| | | Flexibility of cosmetic film | A |

(※16)Trade name KSG-15(Shin-Etsu Chemical Co., Ltd.)
(※17)Trade name SILICONE KF-96A 6CS(Shin-Etsu Chemical Co., Ltd.)
(※18)Trade name AMIHOPE LL(Ajinomoto Co., Inc.)

As apparent from the results shown in Table 5, the water-in-oil emulsion solid base cosmetic of Example 8 was excellent in spreadability, non-stickiness, less feeling in tightness, cosmetic persistence, and flexibility of cosmetic film.

### INDUSTRIAL APPLICABILITY

According to the present invention, there is provided an oil-in-water emulsion cosmetic suitable as a make-up cosmetic or sunscreen cosmetic which is excellent in spreadability, non-stickiness, and flexibility of cosmetic coating film.

1. A water-in-oil emulsion cosmetic comprising:
1 to 20% by mass of trimethylsiloxysilicate that has a viscosity display value of 500 to 130 [(mPa·s)×(g/cm³)] when being dissolved in decamethylcyclopentasiloxane at a concentration of 50% by mass (A);
10 to 60% by mass of a volatile oil (B);
0.2 to 10% by mass of a nonionic surfactant having an HLB of 2 to 8 (C); and
0 to 10% by mass of a nonvolatile liquid oil (D),
wherein a ratio by mass of an amount of the component (A) to a total amount of the components (C) and (D) is 1 to 3.

2. The water-in-oil emulsion cosmetic according to claim 1, wherein the total amount of the components (C) and (D) is 15% by mass or less.

3. The water-in-oil emulsion cosmetic according to claim 1, wherein the component (A) is a mixture of trimethylsiloxysilicate having a viscosity display value of more than 500 [(mPa·s)×((g/cm³)] (X) and trimethylsiloxysilicate having a viscosity display value of less than 130 [(mPa·s)×(g/cm³)] (Y).

4. The water-in-oil emulsion cosmetic according to claim 1, wherein the component (X) has a viscosity display value of 500 to 7,000 [(mPa·s)×(g/cm³)], and the component (Y) has a viscosity display value of 30 to 100 [(mPa·s)×(g/cm³)].

5. The water-in-oil emulsion cosmetic according to claim 3, wherein a ratio by mass of the component (X) to the component (Y), [[(X)/(Y)], is 0.5 to 4.

6. The water-in-oil emulsion cosmetic according to claim 1, further comprising 5 to 80% by mass of water (E).

7. The water-in-oil emulsion cosmetic according to claim 1, wherein the ratio of the component (D) is 0.2 to 7% by mass.

8. The water-in-oil emulsion cosmetic according to claim 1, which is a make-up cosmetic, a sunscreen cosmetic, or a skin care cosmetic.

9. The water-in-oil emulsion cosmetic according to claim 1, wherein the component (A) is trimethylsiloxysilicate having a viscosity display value of 450 to 150 [(mPa·s)×(g/cm³)].

10. The water-in-oil emulsion cosmetic according to claim 1, wherein the ratio by mass of the amount of the component (A) to the total amount of the components (C) and (D) is 1.13 to 3.
